(19) United States
(12) Patent Application Publication
(10) Pub. No.: US 2026/0315069 A1
(43) Pub. Date: Oct. 15, 2026

(54) HANDLE CASE AND SURGICAL INSTRUMENT INCLUDING THE SAME

(71) Applicant: LIVSMED INC., Seongnam-si (KR)

(72) Inventors: Woo Jung CHOI, Siheung-si (KR); Seung Gweon LEE, Suwon-si (KR)

(21) Appl. No.: 19/645,199

(22) Filed: Apr. 12, 2026

(30) Foreign Application Priority Data
Apr. 11, 2025 (KR) 10-2025-0047655

(51) Int. Cl. A61B 17/072; A61B 17/00

(52) U.S. Cl. CPC A61B 17/07207; A61B 2017/00367; A61B 2017/00398; A61B 2017/00477

(57) ABSTRACT

A handle case mountable on a surgical instrument includes a first housing having an accommodation space configured to accommodate a driving module, and an open end through which the driving module enters the accommodation space, a second housing configured to cover the open end of the first housing, and a guide assembly detachably disposed at the open end of the first housing and configured to guide an insertion path of the driving module.

### CROSS-REFERENCE TO RELATED APPLICATION

This application is based on and claims priority under 35 U.S.C. §119 to Korean Patent Application No. 10-2025-0047655, filed on Apr. 11 2025, in the Korean Ministry of Intellectual Property (MOIP) of the Republic of Korea, the disclosure of which is incorporated by reference herein in its entirety.

### BACKGROUND

### 1. Field

The present disclosure relates to a handle case and a surgical instrument including the same.

### 2. Description of the Related Art

Surgery denotes a process of curing illness by cutting, incising, or manipulating the skin, a mucosa layer, and other tissues by using a medical instrument. In particular, laparotomy operations that treat, shape, or remove an organ by cutting and opening the skin of a surgical site may cause bleeding, side effects, pain of a patient, scarring, etc. Therefore, surgery performed by inserting only a medical instrument, e.g., a laparoscope, a surgical instrument, a microscope for microsurgery, etc. after forming a predetermined hole in the skin, or surgery using a robot has been recently considered as an alternative.

A surgical instrument is an instrument for carrying out surgery on a surgical site by manipulating an end tool provided at an end of a shaft that passes through a hole in the skin, manually by a doctor with his/her own hands or by using a robot arm. The end tool provided at the surgical instrument performs a pivoting motion, a gripping motion, a cutting motion, etc. via a predetermined structure.

The above-mentioned background technology is technical information that the inventor possessed for deriving the present disclosure or acquired in the process of deriving the present invention, and cannot necessarily be said to be known art disclosed to the general public before filing the application for the present disclosure.

### SUMMARY

The present disclosure provides a handle case and a surgical instrument, guiding an insertion path of a driving module when laparoscopic surgery or various other surgeries are carried out and having improved convenience in use.

According to an embodiment of the present disclosure, a handle case that may be mounted on a surgical instrument may include a first housing having an accommodation space configured to accommodate a driving module, and an open end through which the driving module enters the accommodation space, a second housing configured to cover the open end of the first housing, and a guide assembly detachably disposed at the open end of the first housing and configured to guide an insertion path of the driving module.

In another embodiment of the present disclosure, the second housing may be rotatably coupled to the first housing.

In the other embodiment of the present disclosure, a stopper protrusion to which either the guide assembly or the second housing may be coupled protrudes from an outer circumferential surface of the first housing.

In the other embodiment of the present disclosure, the first housing is formed as a case having a hollow interior, and may include a raised part protruding outward from a portion of the first housing to define an expanded space.

In the other embodiment of the present disclosure, the first housing may include a housing body in which the driving module may be mounted, and a first handle assembly disposed at a predetermined angle with respect to the housing body and configured to be gripped by a user.

In the other embodiment of the present disclosure, the first handle assembly and the housing body may be disposed to form an obtuse angle.

In the other embodiment of the present disclosure, the first handle assembly may include a first handle extending in the longitudinal direction, and a trigger connected to the first handle, and the trigger may contact a switch disposed on the second housing and configured to generate an electrical signal for driving the driving module.

In the other embodiment of the present disclosure, the trigger may include an input transmission part extending toward the second housing and configured to contact the switch.

In the other embodiment of the present disclosure, the first handle may include an elastic region including an elastic material, and the trigger may include a contact protrusion transmitting the elastic recovery force to the trigger via the contact with the elastic region.

In the other embodiment of the present disclosure, the trigger may include a hook protruding outward and being hooked and coupled to the first handle.

In the other embodiment of the present disclosure, the guide assembly may include a coupling part that may be coupled to the open end of the first housing, and a guide connected to the coupling part and inclined in the direction toward the inside of the first housing.

In the other embodiment of the present disclosure, the coupling part may be press-fitted with the first housing.

In the other embodiment of the present disclosure, the coupling part may have a dual inner wall structure including a first inner wall and a second inner wall that are disposed along a circumference of the coupling part and spaced apart from each other by a predetermined interval, and a portion of the first housing may be inserted between the first inner wall and the second inner wall.

In the other embodiment of the present disclosure, the coupling part may further include a detachment part extending outward.

In the other embodiment of the present disclosure, the second housing may include a cover configured to cover the open end of the first housing, and a second handle assembly extending from the cover and configured to be gripped by the user.

In the other embodiment of the present disclosure, the cover may include a cover body accommodating a printed circuit board therein and connected to the first housing, and an inner case configured to seal the open end of the first housing and contact the driving module.

In the other embodiment of the present disclosure, at least a portion of the inner case may be formed of a material having elastic recovery force.

In the other embodiment of the present disclosure, an outer circumferential surface defined along an edge of the inner case may be an inclined surface disposed to contact an inner circumference of the first housing.

In the other embodiment of the present disclosure, the cover may further include an outer case disposed to face the inner case and having an interface for manipulating the driving of the driving module.

In the other embodiment of the present disclosure, the interface may be configured to operate by receiving the application of power from the driving module, and may convert a manipulation signal that is input from outside into an electrical signal and transmit the electrical signal to the driving module.

In the other embodiment of the present disclosure, the second handle assembly may include a second handle extending in the longitudinal direction, and a second support disposed at a predetermined angle relative to the second handle and having a plate shape.

In the other embodiment of the present disclosure, the second support may extend in a direction away from the first housing.

According to another embodiment of the present disclosure, a surgical instrument including an end tool may include one or more jaws configured to be pivotable, and a manipulation part including a driving module configured to generate power for driving the end tool, and a handle case that accommodates the driving module and may be gripped by a user. The handle case may include a first housing defining an accommodation space for accommodating the driving module and having one open end through which the driving module enters the accommodation space, and a second housing covering the open end of the first housing.

In the other embodiment of the present disclosure, the second housing may be rotatably coupled to the first housing.

In the other embodiment of the present disclosure, a stopper protrusion to which the second housing may be coupled may protrude from the outer circumference of the first housing.

In the other embodiment of the present disclosure, the first housing is formed as a case having a hollow interior, and may include a raised part protruding outward from a portion of the first housing to define an expanded space.

In the other embodiment of the present disclosure, the accommodation space may have a shape corresponding to a cylindrical shape of the driving module that extends longitudinally in one direction, and the accommodation space may be enclosed from the outside when the second housing is coupled to the first housing.

In the other embodiment of the present disclosure, the first housing may include a housing body in which the driving module may be mounted, and a first handle assembly disposed at a predetermined angle with respect to the housing body and configured to gripped by a user.

In the other embodiment of the present disclosure, the first handle assembly and the housing body may be disposed to form an obtuse angle.

In an embodiment of the present disclosure, the first handle assembly may include a first handle extending in the longitudinal direction, and a trigger connected to the first handle, and the trigger may contact a switch disposed on the second housing and configured to generate an electrical signal for driving the end tool.

In the other embodiment of the present disclosure, the trigger may include an input transmission part extending toward the second housing and configured to contact the switch.

In the other embodiment of the present disclosure, the first handle may include an elastic region including an elastic material, and the trigger may include a contact protrusion transmitting the elastic recovery force to the trigger via the contact with the elastic region.

In the other embodiment of the present disclosure, the trigger may include a hook protruding outward and being hooked and coupled to the first handle.

In the other embodiment of the present disclosure, the second housing may include a cover configured to cover the open end of the first housing, and a second handle assembly extending from the cover and configured to be gripped by the user.

In the other embodiment of the present disclosure, the cover may include a cover body accommodating a printed circuit board therein and connected to the first housing, and an inner case configured to seal the open end of the first housing and contact the driving module.

In the other embodiment of the present disclosure, at least a portion of the inner case may be formed of a material having elastic recovery force.

In the other embodiment of the present disclosure, the outer circumferential surface defined along the edge of the inner case may be an inclined surface disposed to contact an inner circumference of the first housing.

In the other embodiment of the present disclosure, the cover may further include an outer case disposed to face the inner case and having an interface for manipulating the driving of the end tool.

In the other embodiment of the present disclosure, the interface is driven by receiving the application of power from the driving module, and the interface may convert a manipulation signal that is input from outside into an electrical signal and transmit the electrical signal to the driving module.

In the other embodiment of the present disclosure, the second handle assembly may include a second handle extending in the longitudinal direction, and a second support disposed at a predetermined angle with respect to the second handle and having a plate shape.

In the other embodiment of the present disclosure, the second support may extend in a direction away from the first housing.

Other aspects, features and advantages other than those described above will become apparent from the following detailed description of the drawings, claims and disclosure.

### BRIEF DESCRIPTION OF THE DRAWINGS

The above and other aspects, features, and advantages of certain embodiments of the disclosure will be more apparent from the following description taken in conjunction with the accompanying drawings.

FIG. 1 is a perspective view illustrating a surgical instrument to which a handle case according to an embodiment of the present disclosure is applied.

FIG. 2 is a perspective view of a reload assembly according to an embodiment of the present disclosure.

FIG. 3 is a perspective view of an end tool according to an embodiment of the present disclosure.

FIGS. 4 and 5 are diagrams illustrating processes of mounting a driving module in a handle case according to an embodiment of the present disclosure.

FIG. 6 is a perspective view of a manipulation part according to an embodiment of the present disclosure.

FIG. 7 is a perspective view of the manipulation part from another direction, according to an embodiment of the present disclosure.

FIG. 8 is a perspective view of a first housing according to an embodiment of the present disclosure.

FIG. 9 is a side view of the first housing according to an embodiment of the present disclosure.

FIG. 10 is a front view of the first housing according to an embodiment of the present disclosure.

FIG. 11 is an enlarged view of part A in FIG. 10, illustrating a state in which a trigger is removed.

FIG. 12 is a front view of the trigger of FIG. 10.

FIG. 13 is a perspective view of the trigger of FIG. 10.

FIG. 14 is an enlarged view of part A in FIG. 10.

FIG. 15 is a diagram showing the trigger coupled to a first handle.

FIG. 16 is a perspective view of a guide assembly according to an embodiment of the present disclosure.

FIG. 17 is a perspective view of the guide assembly from another direction, according to an embodiment of the present disclosure.

FIG. 18 is a cross-sectional view taken along line I-I' of FIG. 4.

FIG. 19 is a perspective view of a second housing according to an embodiment of the present disclosure.

FIG. 20 is a perspective view of the second housing from another direction, according to an embodiment of the present disclosure.

FIG. 21 is a front view of the second housing according to an embodiment of the present disclosure.

FIG. 22 is an exploded perspective view of the second housing according to an embodiment of the present disclosure.

FIG. 23 is an exploded perspective view of the second housing from another direction, according to an embodiment of the present disclosure.

FIG. 24 is a side-sectional view of a manipulation part while a handle case of the present disclosure is in a closed state.

FIG. 25 is an enlarged view of part B of FIG. 24.

### DETAILED DESCRIPTION

The embodiments will be described below in more detail with reference to the accompanying drawings. Those components that are the same or are in correspondence are assigned the same reference numeral regardless of the figure number, and redundant explanations are omitted.

As the present disclosure allows for various changes and numerous embodiments, particular embodiments will be illustrated in the drawings and described in detail in the written description. The attached drawings for illustrating one or more embodiments are referred to in order to gain a sufficient understanding, the merits thereof, and the objectives accomplished by the implementation. However, the embodiments may have different forms and should not be construed as being limited to the descriptions set forth herein.

In the description, certain detailed explanations of the related art are omitted when it is deemed that they may unnecessarily obscure the essence of the present disclosure.

An expression used in the singular encompasses the expression of the plural, unless it has a clearly different meaning in the context. It will be understood that although the terms "first," "second," etc. may be used herein to describe various components, these components should not be limited by these terms. These components are only used to distinguish one component from another. These components are only used to distinguish one component from another.

In the present specification, it is to be understood that the terms such as "including," "having," and "comprising" are intended to indicate the existence of the features or components disclosed in the specification, and are not intended to preclude the possibility that one or more other features or components may exist or may be added.

It will be understood that when a unit, region, or component is referred to as being "formed on" or “disposed on” another layer, region, or component, it can be directly or indirectly formed or disposed on the other layer, region, or component. That is, for example, intervening units, regions, or components may be present.

It will be understood that when an element is referred to as being "connected" or "coupled" to another element, it can be directly connected or coupled to the other element or intervening elements may be present.

Sizes of components in the drawings may be exaggerated for convenience of explanation. In other words, since sizes and thicknesses of components in the drawings are arbitrarily illustrated for convenience of explanation, the following embodiments are not limited thereto.

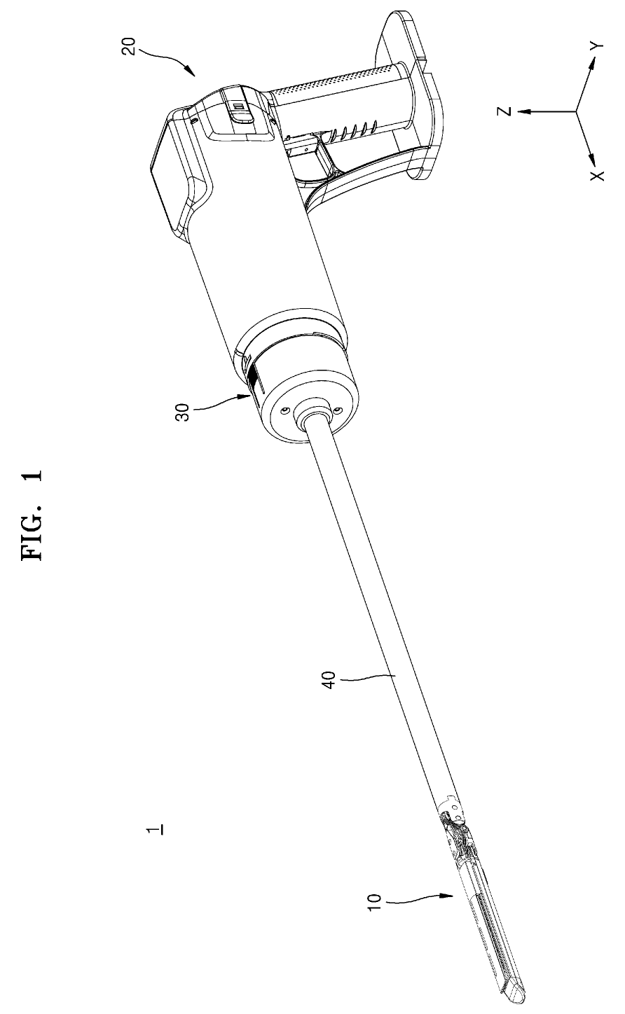

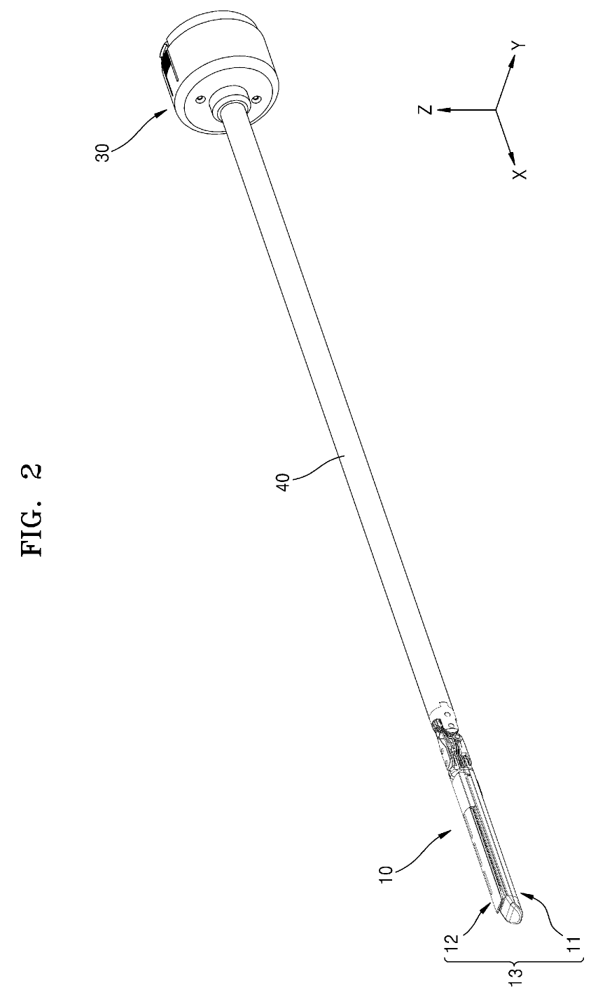

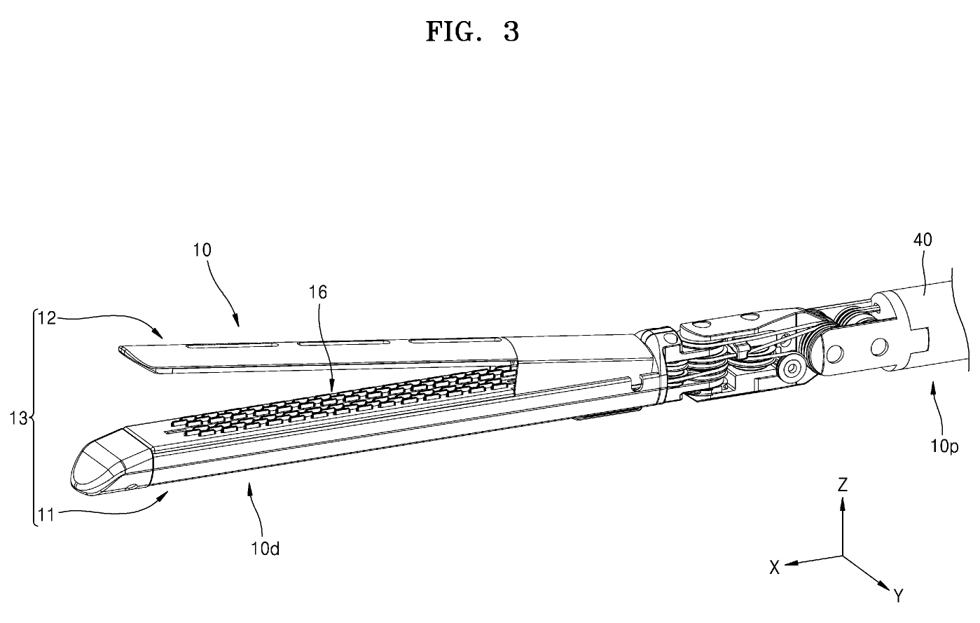

FIG. 1 is a perspective view illustrating a surgical instrument to which a handle case according to an embodiment of the present disclosure is applied. FIG. 2 is a perspective view of a reload assembly according to an embodiment of the present disclosure. FIG. 3 is a perspective view of an end tool according to an embodiment of the present disclosure.

In describing the present disclosure, a part close to a user, that is, a part adjacent to a manipulation part 20, is described as a proximal end, and a part apart from the user, that is, a part adjacent to an end portion of an end tool 10, is described as a distal end.

For example, a part of the end tool 10, which is adjacent to a connection part (shaft) 40, is defined as a proximal end 10p, and a part apart from the connection part (shaft) 40, that is, adjacent to the end portion of the end tool 10, is defined as a distal end 10d.

The end tool 10 according to an embodiment of the present disclosure is connected to one end portion of the connection part (shaft) 40 of a surgical instrument and is inserted into a surgical site to perform motions required for surgery.

First, the surgical instrument and a reload assembly according to an embodiment of the present disclosure are described below.

Referring to FIGS. 1-3, the surgical instrument according to the embodiment may include the end tool 10, the manipulation part 20, the connection part 40, and a power transmission part 30.

In addition, as shown in FIG. 2, the structure including the end tool 10, the connection part (or shaft) 40, and the power transmission part 30 is separately denoted as a reload assembly.

Here, the connection part 40 is formed in the shape of a hollow shaft, and one or more wires and electric wires may be accommodated therein. For convenience of description, the shaft is referred to as the connection part 40 and described. The manipulation part 20 is coupled to one end portion of the connection part 40, the end tool 10 is coupled to the other end portion of the connection part 40, and thus, the connection part 40 may serve to connect the manipulation part 20 to the end tool 10. For example, the connection part 40 may include a straight-line portion, and although not shown in the drawings, may include one or more curved portions for convenience in use and for controlling arrangement of manipulation elements.

The power transmission part 30 is disposed at the other end portion of the connection part 40, and may serve to transfer power generated by a power generator that is described later to the end tool 10. For example, the power transmission part 30 may be arranged between the end tool 10 and the manipulation part 20. As described later, when a user such as a doctor manipulates the manipulation part 20, the power generator generates power for controlling the end tool 10, and the generated power may be transmitted to the end tool 10 via the power transmission part 30. The power transmission part 30 may include a plurality of wires, pulleys, links, sections, gears, etc.

The user may operate the end tool 10 by manipulating the manipulation part 20. For example, the manipulation part 20 is an element for the user to input a signal for controlling motions of the end tool 10. That is, the manipulation part 20 may be considered as an element receiving a signal for controlling motions of the end tool 10 from the user. Here, the signal for controlling the motions of the end tool 10 may be a mechanical manipulation such as a pushing motion of a button or switch, pivoting or movement of a certain member, or an electrical signal generated through the mechanical manipulation, but is not limited thereto.

The manipulation part 20 is provided as an interface that the doctor may directly manipulate, e.g., a gun shape, forceps shape, a stick shape, a lever shape, etc., and when the doctor manipulates the manipulation part, the end tool 10 that is connected to the interface and inserted into the body of a patient performs certain motions to perform surgery.

Here, in FIG. 1, the manipulation part 20 may be formed as a gun shape, but the present disclosure is not limited thereto, that is, the manipulation part of various types that may be connected to the end tool 10 to manipulate the end tool 10 may be possible.

The manipulation part 20 may include a housing defining the exterior of the manipulation part 20. At least a part of the power generator that generates power for controlling the end tool 10 may be accommodated in the housing. Also, a circuit unit for controlling motions of the power generator and a slip ring for supplying electrical energy to the power generator, connecting communication, or transmitting various signals may be accommodated.

The manipulation part 20 may have a handle. The handle is a part that the user grips. Therefore, the user may use the surgical instrument 10 while gripping the handle of the manipulation part 20.

In addition, although not shown in the drawings, a button, a switch, a lever, etc. for controlling various motions of the end tool 10 may be further disposed on the manipulation part.

The end tool 10 is disposed on the other end portion of the connection part 40, and performs necessary motions for surgery after being inserted into a surgical site. As an example of the end tool 10 described above, a pair of jaws 13 for performing a grip motion may be used. However, the concept of the present disclosure is not limited thereto, and various devices for performing surgery may be used as the end tool 10. For example, a configuration of a cantilever cautery may also be used as the end tool. The end tool 10 as above is connected to the manipulation part 20 via the power transmission part 30 (e.g., wire, etc.) and receives the power of the manipulation part 20 through the power transmission part 30, and thus performs a motion necessary for surgery such as gripping, cutting, suturing, or the like.

Hereinafter, the end tool 10 of the surgical instrument of FIG. 3 will be described in more detail.

The end tool 10 may include the jaws 13 and a cartridge 16.

The jaws 13 may perform various functions, e.g., a gripping motion, and in detail, may include a pair of jaws, that is, a first jaw 11 and a second jaw 12. Here, each of the first jaw 11 and the second jaw 12 or a configuration including the first jaw 11 and the second jaw 12 may be referred to as the jaws 13.

The first jaw 11 and the second jaw 12 are arranged to face each other, and may move to be close to or away from each other; for example, the first jaw 11 and the second jaw 12 may be formed to rotationally move about one shaft.

The cartridge 16 may be arranged to be accommodated in the first jaw 11, and a plurality of staples may be arranged in the cartridge 16. While the first jaw 11 and the second jaw 12 are close to each other, for example, while the first jaw 11 and the second jaw 12 are closed with a body tissue interposed therebetween, when an operation member (not shown) receives the force applied thereto, the operation member moves in a direction of the distal end 10d of the first jaw 11 to push the staples and perform stapling operation.

The jaw 13 of the end tool 10 is described in more detail below.

Referring to FIG. 3, the first jaw 11 may be formed as a lengthy bar shape, and a rotary shaft may be arranged at the proximal end 10p to be rotationally moved, and the rotary shaft may correspond to the rotary shaft formed on the second jaw 12 described above. Also, the cartridge 16 may be accommodated in the side closer to the distal end 10d than the rotary shaft.

For example, the first jaw 11 may be formed in a shape of which one surface (upper surface) is removed from an empty box, and a cartridge accommodation part accommodating the cartridge 16 may be defined in the first jaw 11. That is, the first jaw 11 may have a cross-section having a 'U'-shape.

The second jaw 12 may entirely be formed as a lengthy bar shape, for example, the second jaw 12 may be formed as a bar shape corresponding to at least one region of the first jaw 11.

The proximal end 10p of the second jaw 12 may include a region coupled to the first jaw 11. For example, the second jaw 12 is formed to be rotatable about one shaft of the proximal end 10p with respect to the first jaw 11.

The second jaw 12 may have various shapes, for example, from among the surfaces of the second jaw 12, a surface facing the first jaw 11 may have a plurality of anvil grooves in at least one region, and the anvil groove may have a shape corresponding to that of the staple.

The anvil groove of the second jaw 12 may serve as a support for bending the staple, when an operating member pushes the staple upward in the stapling motion.

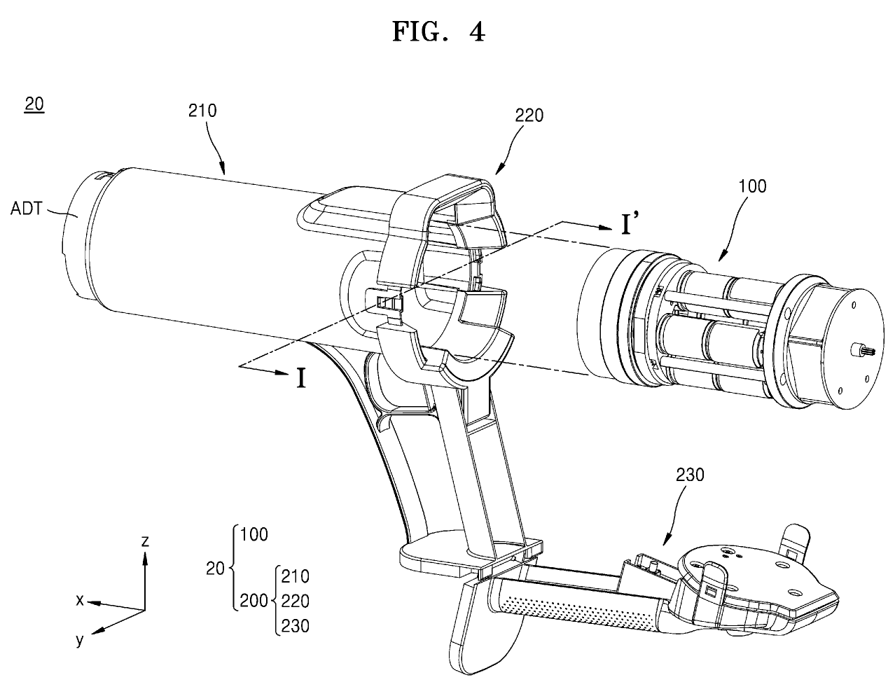

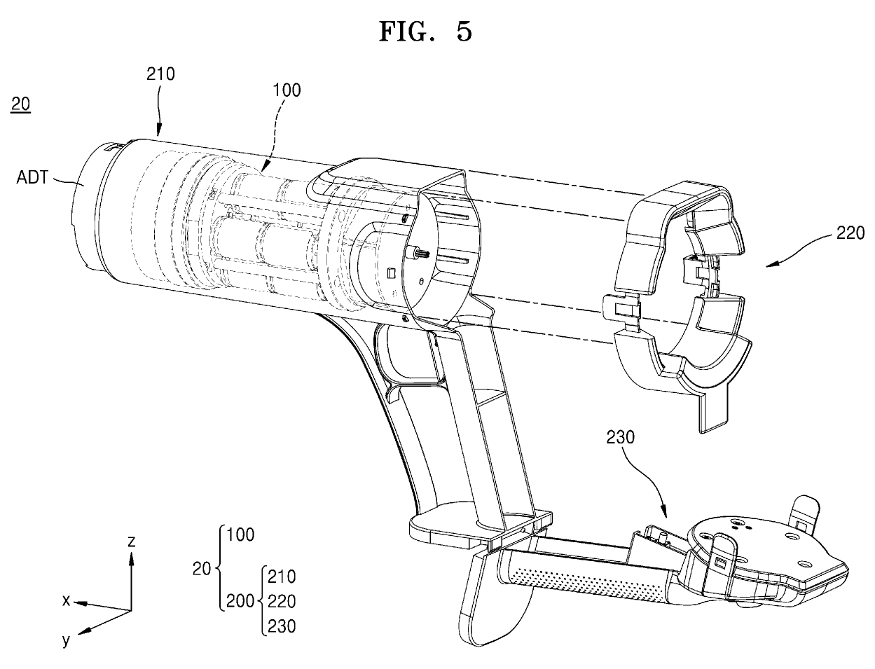

FIGS. 4 and 5 are diagrams illustrating processes of mounting a driving module in a handle case according to an embodiment of the present disclosure.

Referring to FIGS. 4 and 5, the manipulation part 20 may include a driving module 100 and a handle case 200.

The driving module 100 according to an embodiment of the present disclosure generates power for driving the end tool 10, and may be mounted in the handle case 200 that the user may directly manipulate.

The driving module 100 may include at least one motor. For example, the driving module 100 may include at least one motor generating the power for driving the end tool 10 based on a signal input into the handle case 200.

Additionally, the driving module 100 may include a battery, an electronic device, etc. and may control reloading by receiving a user input.

For example, the driving module 100 of the surgical instrument may be formed to be attachable to/detachable from the handle case 200. In this case, the driving module 100 of the surgical instrument may include a module body, and the module body may have a coupling structure to be coupled to the handle case 200.

As described above, when the driving module 100 of the surgical instrument is formed to be attachable to/detachable from the handle case 200, the user may easily replace the driving module 100 as necessary.

The driving module 100 may be connected to the power transmission part 30 while being mounted in the handle case 200 and may generate the power for driving the end tool 10, and the handle case 200 may provide a space in which the driving module 100 may be accommodated. Also, the handle case 200 may receive an input of a manipulation signal from the user and transmit the manipulation signal to the end tool 10.

In other words, the user may drive the driving module 100 by transmitting an input signal to the driving module 100, and the end tool 10 may be driven after receiving the power from the driving module 100 according to the input signal input from the user.

The handle case 200 accommodates the driving module 100 and may be gripped by the user when driving the surgical instrument 1, and may be detachably disposed to be attachable to and detachable from the reload assembly.

A separate adaptor ADT structure for transmitting the power generated from the driving module 100 to the reload assembly may be provided at one end portion of the handle case 200.

In general, the driving module 100 includes a multiple-use product that may be used hundreds of times or more, and when the driving module 100 is used multiple times, the driving module 100 may be exposed to the external environment and contaminated.

The handle case 200 according to an embodiment of the present disclosure is provided in a sterilized state and surrounds the driving module 100 that is in a non-sterilized state, to form a sterilized environment.

Additionally, a handle that may be gripped by the user and an input device (joystick, button, etc.) capable of transmitting the input signal for driving the end tool 10 may be provided in the handle case 200.

The input signal of the user input through the input device of the handle case 200 may be processed by a microcontroller unit (MCU) arranged inside the handle case 200. The input signal may be transmitted to the driving module 100 via pogo pins that may come into contact with the driving module 100. Detailed descriptions thereof are provided later.

The handle case 200 according to an embodiment of the present disclosure may include a first housing 210, a guide assembly 220, and a second housing 230.

The first housing 210 includes an accommodation space for accommodating the driving module 100, and the driving module 100 may enter the accommodation space through an open end of the first housing 210.

The driving module 100 according to an embodiment of the present disclosure may have a cylindrical shape that is lengthily extended in one direction, and the accommodation space may have a shape corresponding to the driving module 100.

The driving module 100 may have a cylindrical shape having one extended shaft, and the accommodation space may be a cylindrical-shaped space having one extended shaft. When the driving module 100 is mounted in the first housing 210, the extended shaft of the driving module 100 and the extended shaft of the accommodation space may be placed on the same straight line. In other words, the two extended shafts may match with each other.

When the second housing 230 is coupled to the first housing 210, the accommodation space may be enclosed and isolated from the outside. In other words, the accommodation space may be completely sealed by the second housing 230 and may not be in communication with the space inside the second housing 230.

The second housing 230 may cover the open end of the first housing 210, and at the other side of the second housing 230, which faces one side covering the first housing 210, an input device capable of transmitting the input signal for driving the end tool 10 from the user may be provided.

The first housing 210 and the second housing 230 may be rotatably connected. Specifically, end portions of the first housing 210 and the second housing 230 (lower side in FIG. 4) may be connected to each other in a hinged manner, and the other end portions of the first housing 210 and the second housing 230 (upper side in FIG. 4) facing the end portions may have latch structures that may be detached as necessary.

For convenience of description, the case in which coupling between the upper end portions of the first housing 210 and the second housing 230 is released (FIG. 4) is defined as an 'open state' of the handle case 200, and the case in which the upper end portions of the first housing 210 and the second housing 230 (FIG. 5) are coupled is defined as a 'closed state' of the handle case 200.

That is, the driving module 100 may be mounted in the first housing 210 while the handle case 200 is in the open state. When the mounting of the driving module 100 is finished, the handle case 200 may be in the closed state provided that the second housing 230 is coupled to the open end of the first housing 210. As such, the driving module 100 that is not sterilized may be sealed by the handle case 200.

The guide assembly 220 according to an embodiment of the present disclosure may be arranged to be attachable to/detachable from the open end of the first housing 210 and guides an insertion path of the driving module 100, and may be mounted to the first housing 210 while the handle case 200 is in the open state.

While the guide assembly 220 is mounted in the first housing 210, the driving module 100 may be inserted into the first housing 210.

The guide assembly 220 may have both sides that are open. That is, the guide assembly 220 may have a shape having a hollow center, and areas of the both open ends of the guide assembly 220 may be different from each other.

The guide assembly 220 may have an inclined surface in a direction from the outside to the inside of the first housing 210. Here, an area of the opened region of the guide assembly 220 may be gradually reduced in a direction from the outside to the inside of the first housing 210.

The guide assembly 220 may allow the inlet in which the driving module 100 is inserted to be gradually narrowed and guide the insertion path so that the mounting of the driving module 100 may be easily performed.

Also, a center of the driving module 100 in the axial direction and a center of the first housing 210 in the axial direction may be naturally aligned, and thus, the driving module 100 may be mounted to a precise position.

The handle case 200 has to be maintained in the sterilized state during surgery. However, the driving module 100 is in generally non-sterilized state, and thus, there is a concern that the handle case 200 may be contaminated due to the contact during the insertion of the driving module 100.

The guide assembly 220 according to an embodiment of the present disclosure guides, when the driving module 100 is inserted into the handle case 200, the insertion path of the driving module 100 so that the center of the driving module 100 and the center of the handle case 200 are aligned with each other, and thus, the driving module 100 may be prevented from coming into contact with the other region than the inside of the handle case 200.

In an embodiment, the handle case 200 may be provided with the guide assembly 220 mounted on the first housing 210 in advance. When the guide assembly 220 is provided while being mounted on the first housing 210, the user may directly insert the driving module 100 into the handle case 200 without an additional assembling process. As such, an issue where the guide assembly 220 is wrongly mounted or the handle case 200 is contaminated during the mounting process may be prevented, and thus, the convenience of using the surgical instrument 1 may be improved.

As shown in FIG. 5, when the mounting of the driving module 100 is finished, the guide assembly 220 may be separated from the open end of the first housing 210.

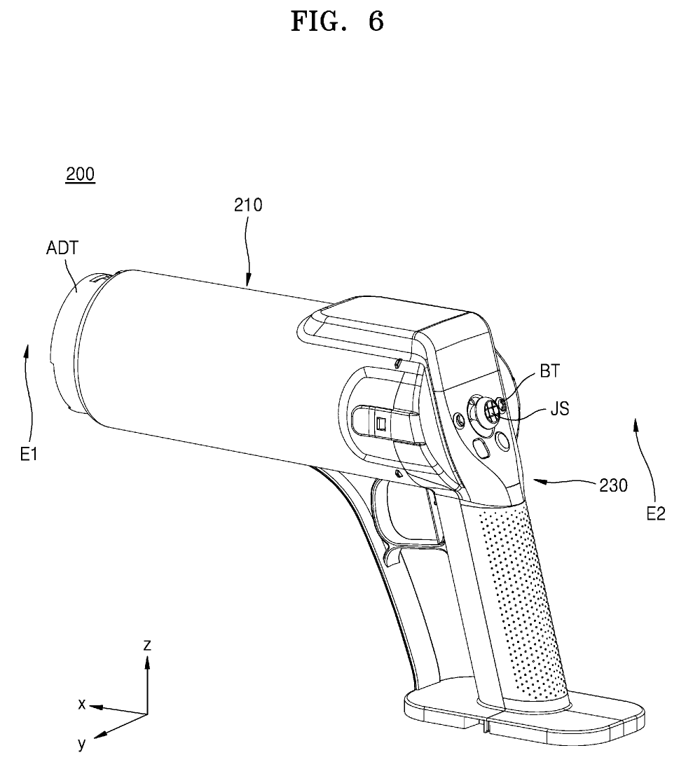

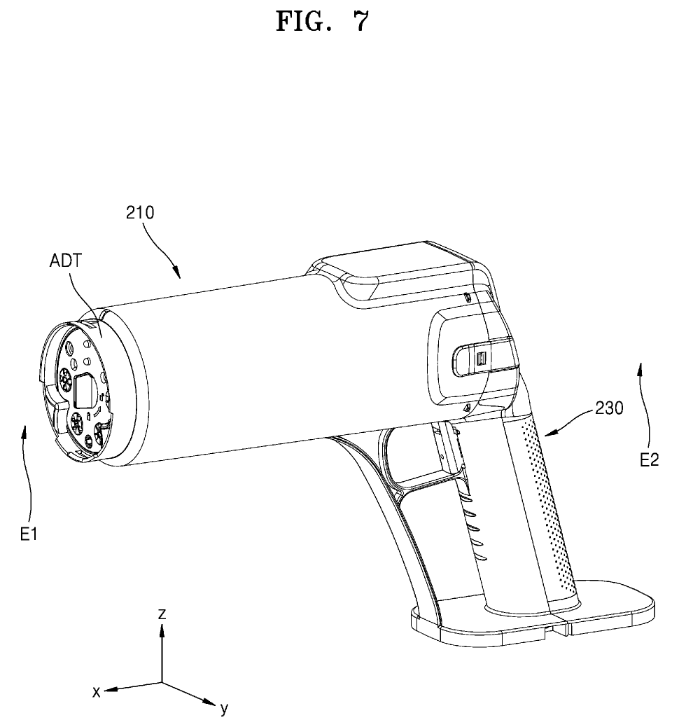

FIG. 6 is a perspective view of a manipulation part according to an embodiment of the present disclosure. FIG. 7 is a perspective view of a manipulation part from another direction, according to an embodiment of the present disclosure.

Referring to FIGS. 6 and 7, after the guide assembly 220 is separated from the first housing 210, the second housing 230 may be coupled to the open end of the first housing 210. The driving module 100 in the non-sterilized state may be completely surrounded by the handle case 200 of sterilized state.

The first housing 210 and the second housing 230 may define the exterior of the manipulation part 20. In an embodiment, the exterior of the manipulation part 20 may have a gun shape.

When the first housing 210 and the second housing 230 are coupled to each other, the handle portion that the user may grip may be formed as a cylindrical shape. The user may control various motions of the end tool 10 by using a button, a switch SW, a lever, etc. while gripping the handle.

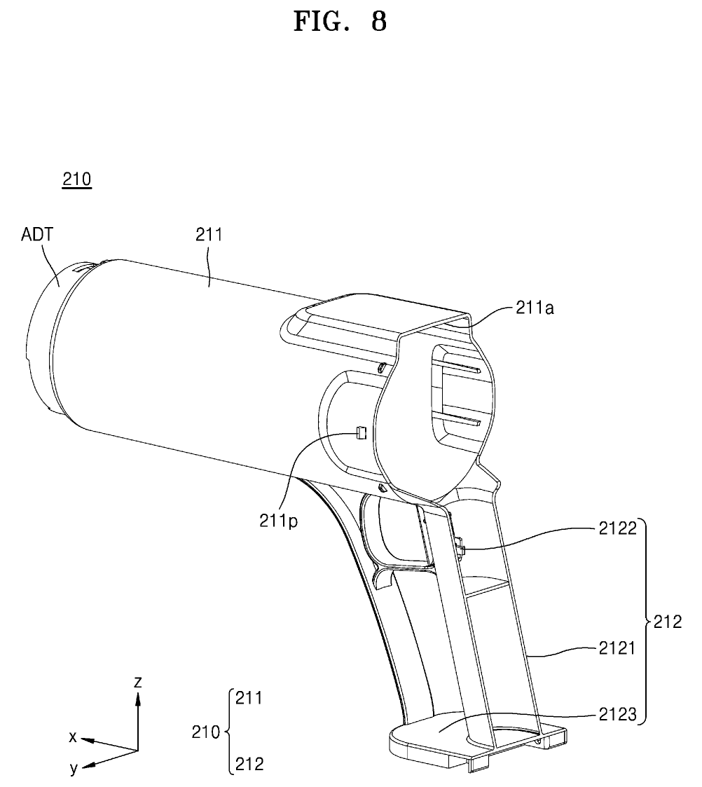

For example, the user may control the proceeding motion of the end tool 10 by pulling a trigger 2122 (see FIG. 8) that is described later and may implement tilting and pivoting motions of the end tool 10 by physically tilting or pivoting a joystick JS.

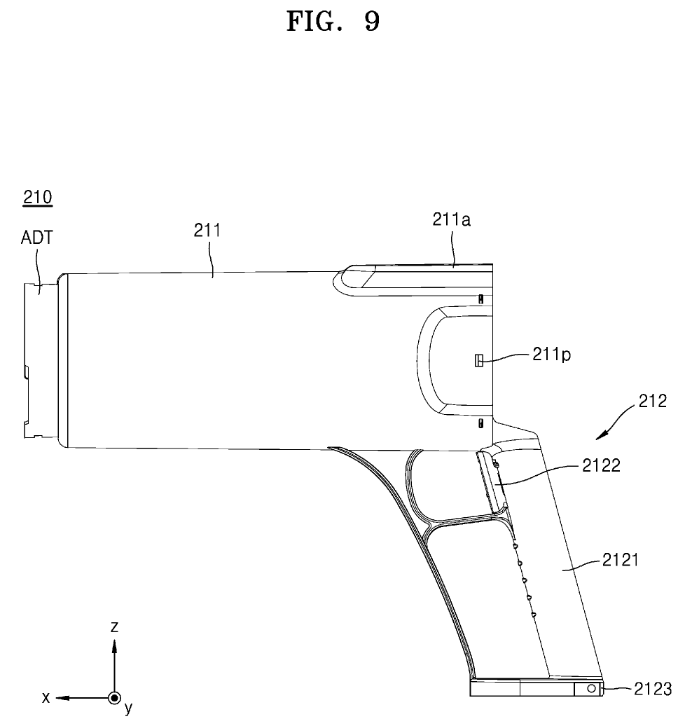

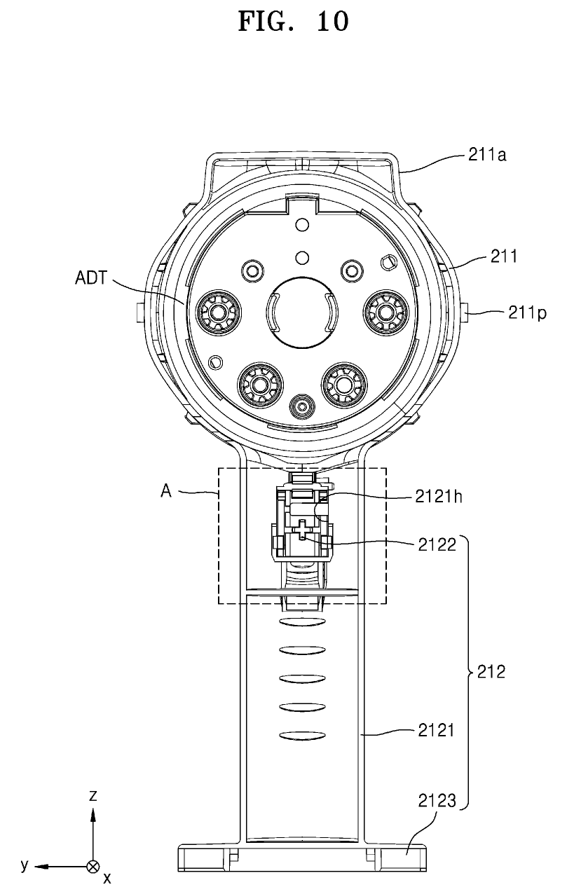

FIG. 8 is a perspective view of a first housing according to an embodiment of the present disclosure. FIG. 9 is a side view of a first housing according to an embodiment of the present disclosure. FIG. 10 is a front view of a first housing according to an embodiment of the present disclosure.

Referring to FIGS. 8-10, the first housing 210 may have the accommodation space for accommodating the driving module 100 and may include a housing body 211 and a first handle assembly 212. The driving module 100 may enter the accommodation space through the open end of the first housing 210.

The driving module 100 may be mounted in the housing body 211 according to an embodiment of the present disclosure. The adaptor ADT may be installed on one side of the housing body 211 and the second housing 230 may be coupled to the other side of the housing body 211, which faces the adaptor ADT. The adaptor ADT is connected to the reload assembly and may transmit the power generated from the driving module 100 to the end tool 10.

The housing body 211 may be formed of a transparent or semi-transparent material. As such, when the driving module 100 is mounted in the housing body 211, a display of the driving module 100 may be recognized from the outside.

The housing body 211 may be formed as a case with hollow interior. For example, the housing body 211 may be formed as a cylindrical case with hollow interior. However, the present disclosure is not limited thereto, and the housing body 211 may be provided as a case of various shapes within the technical idea of accommodating the driving module 100.

The housing body 211 may have a raised part 211a that protrudes from a partial region thereof to define an expanded space. The raised part 211a may be arranged to be closer to one side E2 of the housing body 211, which is covered by the second housing 230, than the other side E1 of the housing body 211, which is coupled to the adaptor ADT, of the housing body 211 covered by the second housing 230.

Also, the raised part 211a may be connected to the side E2 that is open in the housing body 211 and may have one open end.

When the driving module 100 is mounted in the first housing 210, the display of the driving module 100 may be seated in the expanded space defined by the raised part 211a. The raised part 211a serves as a stopper preventing the driving module 100 substantially having the cylindrical shape from moving, so as to fix the driving module 100 not to rotate.

A stopper protrusion 211p, to which one of the guide assembly 220 and the second housing 230 may be coupled, may protrude from the outer circumference of the first housing 210, in detail, the housing body 211.

When the handle case 200 is in the open state, the guide assembly 220 may be coupled to the stopper protrusion 211p, and when the handle case 200 is in the closed state, the second housing 230 may be coupled to the stopper protrusion 211p.

A plurality of stopper protrusions 211p may be provided to be arranged respectively on both sides of the housing body 211. As such, the rotation of the guide assembly 220 or the second housing 230 coupled to the first housing 210 may be prevented.

The first handle assembly 212 according to an embodiment of the present disclosure may be arranged to have an angle set in advance with respect to the housing body 211 and may be gripped by the user, and may include a first handle 2121, a trigger 2122, and a first support 2123.

The first handle assembly 212, e.g., the first handle 2121, and the housing body 211 may be arranged to form an obtuse angle.

The first handle 2121 may extend in the longitudinal direction. The first handle 2121 may extend downward (based on FIG. 9), and may be arranged to have an angle that is at least 90° or greater with respect to the central axis direction of the housing body 211.

The first handle 2121 may be a region directly gripped by the user when driving the surgical instrument 1.

The first handle 2121 may be formed as a half of a hollow cylinder cut in halves. The first handle 2121 may define a cylindrical shape by coupling to a second handle 2321 that is described later. The cylindrical shape may be arranged to be inclined so as to form an angle set in advance with respect to the first support 2123, and the user may easily grip the handle case 200 while surrounding the cylindrical shape formed by the first handle 2121 and the second handle 2321.

The cylindrical shape may be arranged to have an angle of at least 90° or greater with respect to the central axis direction of the housing body 211. Referring back to FIGS. 7 and 8, the first handle 2121 and the second handle 2321 may be arranged in a direction toward one side E2 of the handle case 200.

In other words, the first handle 2121 and the second handle 2321 may be arranged closer to the body of the user, that is, at the outermost side of the surgical instrument 1, while the user grips the handle case 200. As such, the user may manipulate the interface such as the joystick JS, a button BT, etc. easily by the thumb, while gripping the first handle 2121 and the second handle 2321.

A plurality of protrusions (not indicated by reference numerals) protruding outward may be formed on the surface of the first handle 2121. The plurality of protrusions may be arranged to be spaced apart from one another with preset intervals, and provide the user with stabilized grip to prevent slipping when being gripped.

The trigger 2122 according to an embodiment of the present disclosure is connected to the first handle 2121, is arranged on the second housing 230, and may come into contact with the switch SW generating an electrical signal for driving the end tool 10.

The trigger 2122 may be arranged so as to at least partially pass through the first handle 2121. The first handle 2121 facing the trigger 2122 may have one surface having a through-hole 2121h through which at least a part of the trigger 2122 may pass.

At least a part of the trigger 2122 may be arranged in the through-hole 2121hand may be moved within a certain angle range based on the rotating center axis set in advance.

For example, when the user has the finger on the trigger 2122 and pulls the trigger 2122 with the certain force, the trigger 2122 moves in a direction toward the second housing 230 and exerts force to the switch SW arranged on the second housing 230, and the switch SW may generate the electrical signal for driving the end tool 10 when pushed.

When the user releases the trigger 2122, that is, removes the force exerted on the trigger 2122, the trigger 2122 may return to an original position by receiving the elastic recovery force from the first housing 210, and the pushed state of the switch SW may be also released.

Hereinafter, detailed configurations and connection structure of the first handle 2121 and the trigger 2122 are described in detail below.

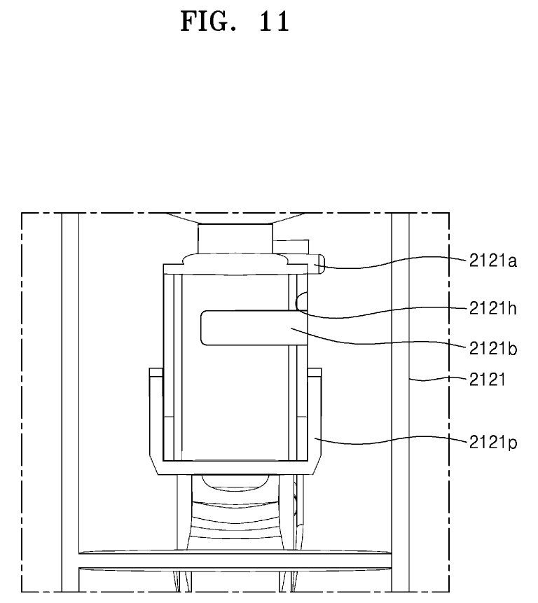

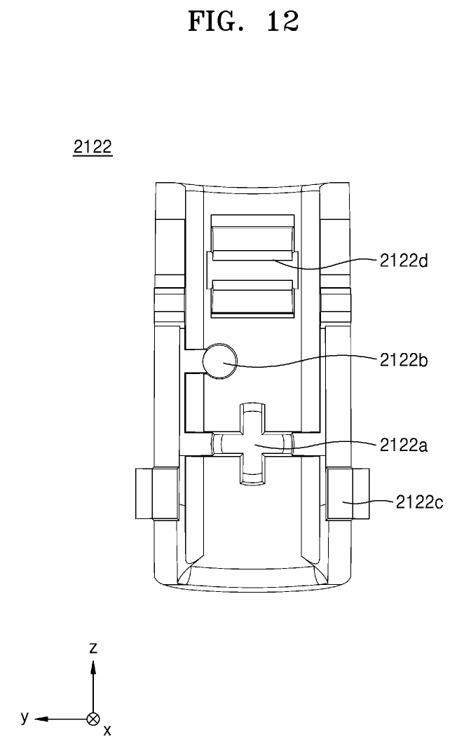

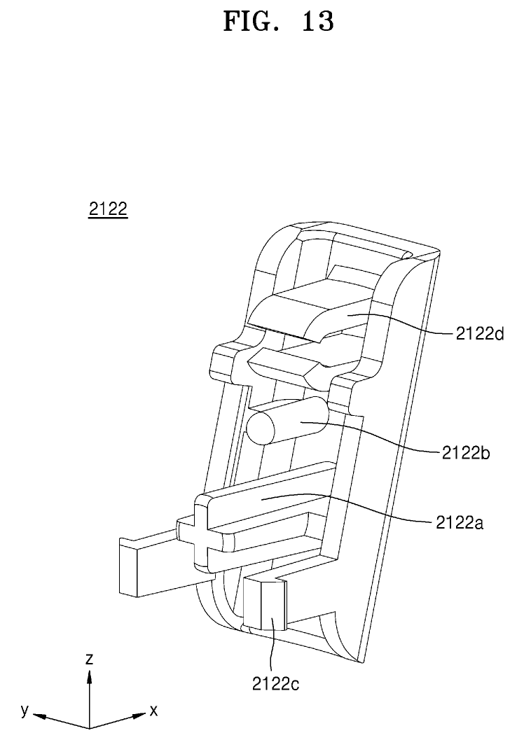

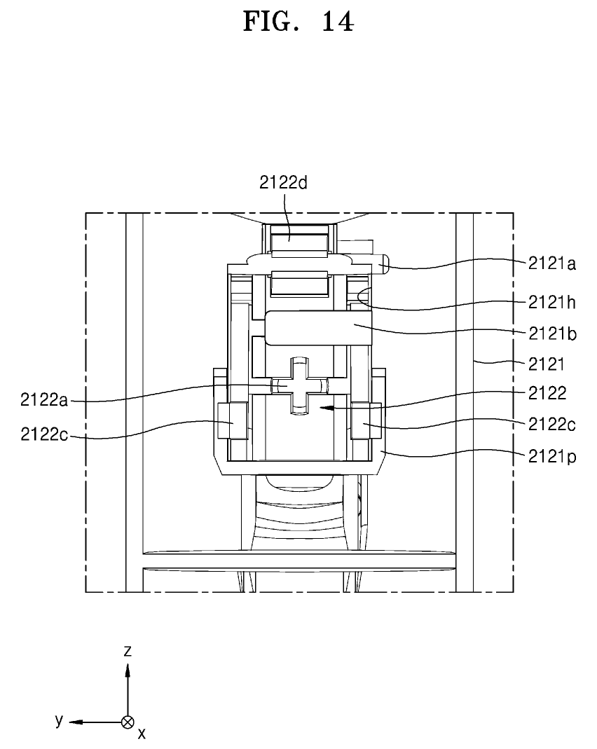

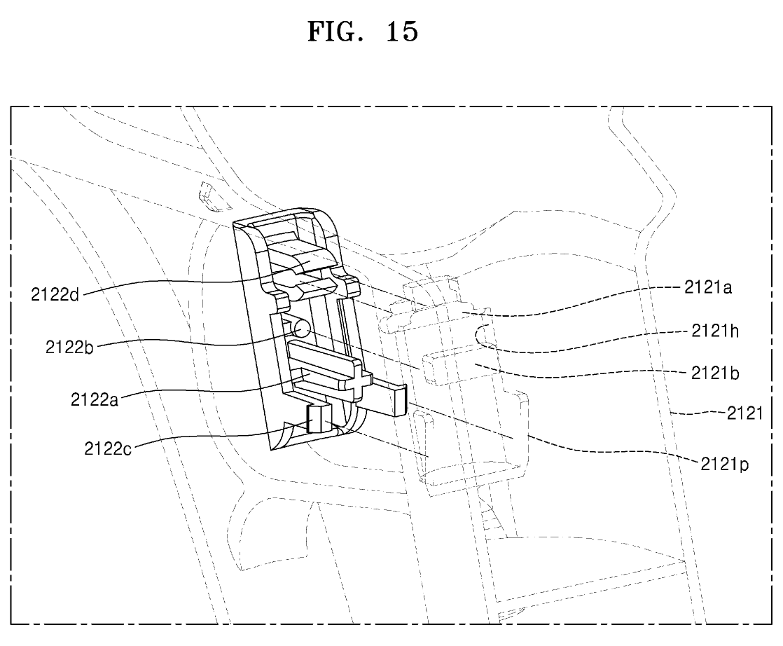

FIG. 11 is a diagram showing a state in which a trigger is removed from an enlarged view of part A in FIG. 10. FIG. 12 is a front view of the trigger of FIG. 10. FIG. 13 is a perspective view of the trigger of FIG. 10. FIG. 14 is an enlarged view of part A in FIG. 10. FIG. 15 is a diagram showing the trigger coupled to a first handle.

Referring back to FIGS. 10 and 11, the first handle 2121 may have the through-hole 2121h, a hinge pin 2121a, an elastic region 2121b, and a protrusion 2121p.

The through-hole 2121h according to an embodiment of the present disclosure may be formed as a hole allowing the inside and the outside of the first handle 2121 to be in communication with each other. In detail, the through-hole 2121h may be formed as a shape corresponding to the trigger 2122, for example, when the trigger 2122 has a rectangular cross-section, the through-hole 2121h may also have a rectangular hole shape to correspond to the cross-sectional shape.

The hinge pin 2121a according to an embodiment of the present disclosure is arranged at one side (upper side in FIG. 11) of the through-hole 2121h, and the trigger 2122 may be connected to the first handle 2121 in a hinged manner via the hinge pin 2121a. The hinge pin 2121a may have a cylindrical shape or shaft shape extending in the longitudinal direction and may act as the rotating center axis of the trigger 2122.

That is, one side of the trigger 2122 may be rotatably coupled to the hinge pin 2121a, and the trigger 2122 may be moved within an angle range set in advance based on the hinge pin 2121a as the rotating center axis.

The elastic region 2121b according to an embodiment of the present disclosure is formed of an elastic material and may protrude toward the inside of the through-hole 2121h. The elastic region 2121b may extend in the width direction of the through-hole 2121h.

The elastic region 2121b may be arranged to be able to come into contact with a contact protrusion 2122b of the trigger 2122, which is described later. When the user has the finger on the trigger 2122 and then pulls the trigger 2122 with certain force, the contact protrusion 2122b may exert force to the elastic region 2121b, and the elastic region 2121b may be partially deformed in the direction of receiving the force, that is, the direction toward the second housing 230.

When the user removes the force exerted on the trigger 2122, the elastic region 2121b may apply an elastic recovery force to the contact protrusion 2122b while returning back to the original position. The elastic recovery force may be transmitted to the trigger 2122 via the contact protrusion 2122b, and the trigger 2122 also returns back to the original position.

The protrusion 2121p according to an embodiment of the present disclosure protrudes in the direction toward the first handle 2121 while surrounding at least a part of the through-hole 2121h, and the trigger 2122 may be latched and coupled to the protrusion 2121p.

The protrusion 2121p may be formed to surround the lower side (based on FIG. 11) of the through-hole 2121h. As such, the trigger 2122 may be respectively coupled to the hinge pin 2121a and the protrusion 2121p at both sides of the through-hole 2121h.

Referring to FIGS. 12 and 13, the trigger 2122 is arranged through the through-hole 2121h of the first handle 2121 and may be rotatably connected to the first handle 2121.

The trigger 2122 according to an embodiment of the present disclosure may extend in the longitudinal direction of the first handle 2121 and may have an input transmission part 2122a, a contact protrusion 2122b, a hook 2122c, and hinge parts 2122d.

The input transmission part 2122a extends in the direction toward the second housing 230 and may have a cross-section having a substantially "+" shape.

The input transmission part 2122a may come into contact with the switch SW arranged inside the second housing 230 when the handle case 200 is in the closed state.

The contact protrusion 2122b may come into contact with the elastic region 2121b of the first handle 2121 and extend in the direction toward the first handle 2121, and may transmit the elastic recovery force to the trigger 2122 via the contact with the elastic region 2121b. One end portion of the contact protrusion 2122b coming into contact with the elastic region 2121b may be formed as a dome shape.

The hooks 2122c protrude outward and are latched and coupled to the first handle 2121, and a plurality of hooks 2122c may be respectively arranged on both sides of the trigger 2122. The hook 2122c may be formed approximately as a hook shape.

At least a part of the hook 2122c may extend toward the first handle 2121 and one end portion of the extended hook 2122c may protrude outward. The protruding end portion of the hook 2122c may be latched and coupled to the protrusion 2121p of the first handle 2121. As such, during the movement of the trigger 2122 with respect to the first handle 2121, the separation or escape of the trigger 2122 from the through-hole 2121h may be prevented.

The hinge parts 2122d are formed to surround the hinge pin 2121a and may extend toward the first housing 210. The hinge parts 2122d may be spaced apart from each other in the longitudinal direction of the trigger 2122 and may respectively surround some parts of the upper and lower sides of the hinge pin 2121a (based on FIG. 14).

That is, the hinge part 2122d and the hinge pin 2121a are coupled in a hinged manner, and the trigger 2122 may rotatably move with respect to the first handle 2121 about the hinge pin 2121a as the rotating center axis.

The first support 2123 is connected to the first handle 2121 and may be formed as a flat plate shape.

The first support 2123 may be arranged on the same plane as the second support 2323 when the handle case 200 is in the closed state. The first support 2123 may support the edge of the hand of the user and provides the user with a stabilized feeling, when the user grips the handle case 200.

Referring to FIGS. 14 and 15, the trigger 2122 may be coupled to the first handle 2121 while passing through the first handle 2121.

The hinge pin 2121a and the elastic region 2121b formed on the first handle 2121 may be arranged to respectively face the hinge parts 2122d and the contact protrusion 2122b disposed on the trigger 2122. The hinge pin 2121a may be coupled to the hinge parts 2122d in a hinged manner, and the elastic region 2121b may be arranged to be capable of contacting the contact protrusion 2122b.

The input transmission part 2122a may pass through the through-hole 2121h, and the hooks 2122c may be latched and coupled to the protrusions 2121p protruding from both sides of the through-hole 2121h.

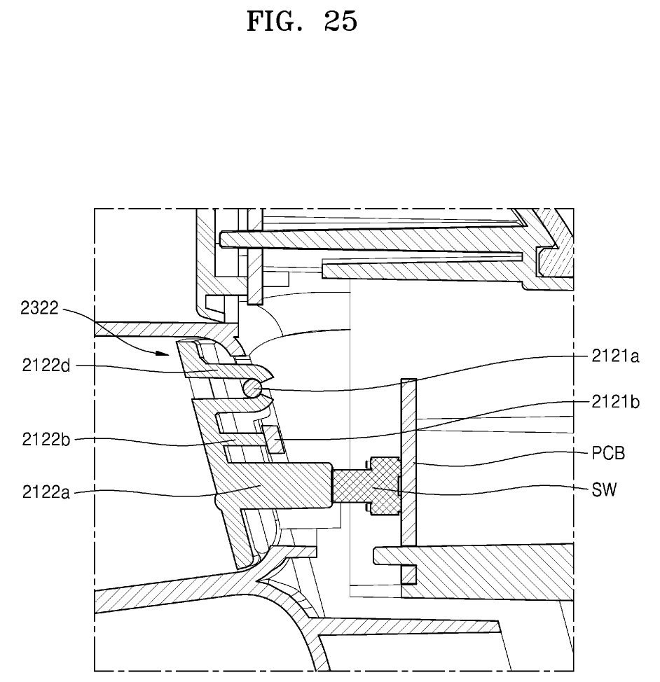

Referring to FIG. 25 that is described later, when the user pulls the trigger 2122 while the input transmission part 2122a and the switch SW are in contact with each other, the input transmission part 2122a may push the switch SW, and the switch SW may generate an electrical signal for driving the end tool 10 and transmit the electrical signal to the driving module 100.

Here, the trigger 2122 may rotate within an angle range set in advance about the hinge pin 2121a as the rotating center axis, and when the trigger 2122 moves in a direction toward the second housing 230, the contact protrusion 2122b may push and deform the elastic region 2121b.

When the external force from the user is removed, the elastic recovery force of the elastic region 2121b may be transmitted to the trigger 2122 via the contact protrusion 2122b, and the trigger 2122 may be recovered to the original position. As such, the force of the input transmission part 2122a for pushing the switch SW may be also removed, and the operation of the switch SW for generating the electrical signal for driving the end tool 10 may be also stopped.

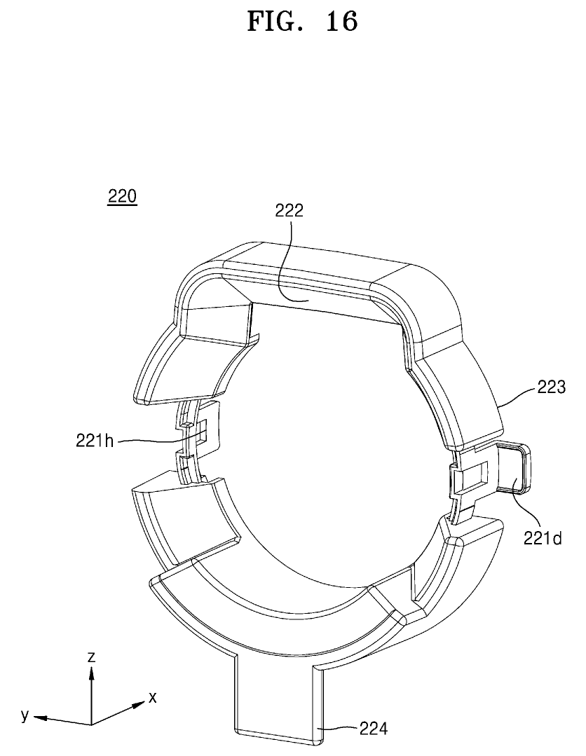

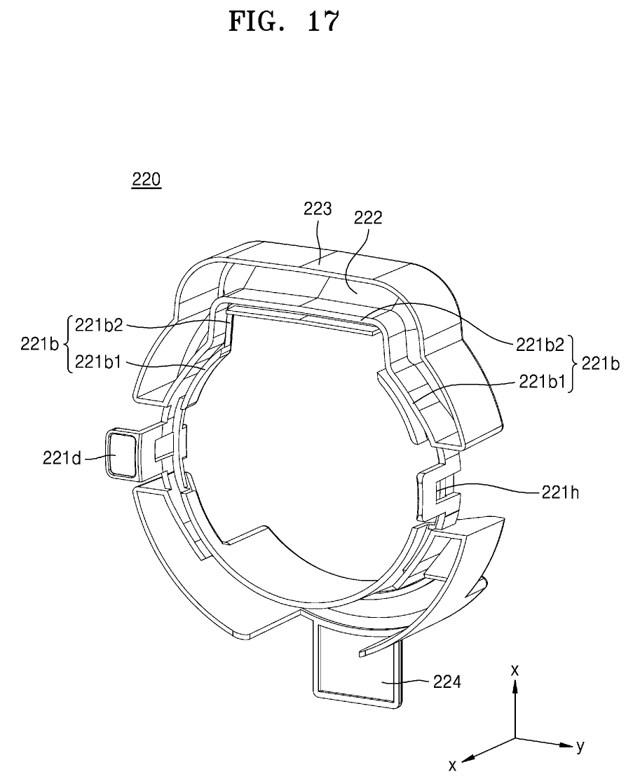

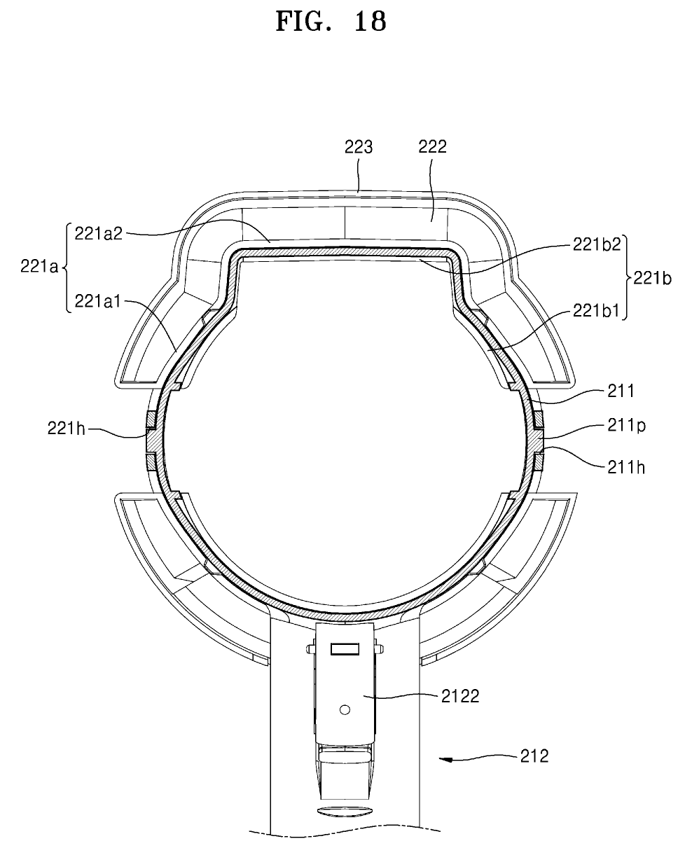

FIG. 16 is a perspective view of a guide assembly according to an embodiment of the present disclosure. FIG. 17 is a perspective view of a guide assembly from another direction, according to an embodiment of the present disclosure. FIG. 18 is a cross-sectional view taken along line I-I' of FIG. 4.

Referring to FIGS. 16 and 17, the guide assembly 220 may be arranged to be attachable to/detachable from the open end of the first housing 210 and guide the insertion path of the driving module 100, and may include a coupling part 221, a guide 222, an outer circumference part 223, and a blocking part 224.

The coupling part 221 according to an embodiment of the present disclosure may be coupled to one open end of the first housing 210, and may include a first inner wall 221a, a second inner wall 221b, a stopper hole part 221h, and a detachment part 221d.

The coupling part 221 may be fitted with the first housing 210 and may have a dual-inner wall structure including the first inner wall 221a and the second inner wall 221b that are arranged along the circumference of the coupling part 221 and spaced apart from each other with a certain gap.

In detail, a part of the first housing 210 may be inserted between the first inner wall 221a and the second inner wall 221b. That is, a gap between the first inner wall 221a and the second inner wall 221b may be equal to or relatively greater than a thickness of the first housing 210.

The coupling part 221 may be formed to have a shape corresponding to the open end of the first housing 210. At least a part of the coupling part 221 may be circular, and a partial region of the coupling part 221 may protrude outward.

In other words, the coupling part 221 may have flat portions 221a2 and 221b2 that are formed in sections set in advance along the outer circumference based on the center axis of the housing body 211 in the longitudinal direction and curved portions 221a1 and 221b1 that are connected to both sides of the flat portions 221a2 and 221b2.

The flat portions 221a2 and 221b2 may be formed as planar shapes. That is, based on the center axis of the first handle 2121 in the longitudinal direction, planes may be formed in the sections set in advance along the inner circumference, and curved surfaces may be formed on both sides of the planes so as to surround the housing body 211.

The raised part 211a may be inserted between the flat portion 221a2 and the flat portion 221b2, and the open end of the first housing 210, except the raised part 211a, may be inserted between the curved portion 221a1 and the curved portion 221b1.

As such, while the guide assembly 220 is coupled to be engaged with the first housing 210, the rotation of the guide assembly 220 with respect to the first housing 210 may be prevented.

Stopper hole parts 221h may protrude toward the first housing 210 and may be coupled to the stopper protrusion 211p, and there may be a plurality of stopper hole parts 221h.

The plurality of stopper hole parts 221h are arranged on both sides of the double-inner wall structure, and may be respectively coupled to the stopper protrusions 211p disposed on the outer circumference of the first housing 210.

Because the double-inner wall structure is fitted with the first housing 210 and the plurality of stopper hole parts 221h are latched and coupled to the first housing 210, the position of the guide assembly 220 may stably be fixed to the first housing 210.

The detachment part 221d is connected to the stopper hole part 221h and extends outward, and may be arranged with an angle set in advance with respect to the stopper hole part 221h. In an embodiment, the angle formed by the stopper hole part 221h and the detachment part 221d may be a right angle.

The detachment part 221d may be connected to one of the plurality of stopper hole parts 221h, but is not limited thereto. That is, a plurality of detachment parts 221d may be provided to be respectively connected to the plurality of stopper hole parts 221h.

When the mounting of the driving module 100 into the first housing 210 is finished, the user may separate the guide assembly 220 from the first housing 210 by pulling the detachment part 221d. When the user exerts the external force to the detachment part 221d in a direction toward the second housing 230, the latch-coupling structure between the stopper hole part 221h and the stopper protrusion 211p may be released, and the guide assembly 220 may be easily separated from the first housing 210.

The detachment part 221d according to an embodiment of the present disclosure may enable the first housing 210 and the guide assembly 220 to be separated with less effort, and thus, the convenience in use of the handle case 200 may be improved.

The guide 222 according to an embodiment of the present disclosure may be connected to the coupling part 221 and may be inclined in the direction toward the inside of the first housing 210.

The outer circumference part 223 according to an embodiment of the present disclosure may be connected to a side of the guide 222, which is opposite to the side connected to the coupling part 221, and may surround the coupling part 221.

The blocking part 224 according to an embodiment of the present disclosure protrudes from the guide 222 to one side (lower side in FIG. 17), and at least a part of the blocking part 224 may be arranged on the first housing 210, specifically, on the first handle 2121.

Referring to FIG. 4, a width of the blocking part 224 may be equal to or less than the width of the first handle 2121 and may cover one side of the through-hole 2121h and the trigger 2122 while the guide assembly 220 is mounted on the first housing 210. As such, the rotation of the guide assembly 220 may be prevented.

Referring back to FIGS. 16-18, the guide 222 may be arranged between the coupling part 221 and the outer circumference part 223, and the coupling part 221 of the double-inner wall structure may be arranged on one side of the guide 222 and the outer circumference part 223 may be arranged on the other side of the guide 222, opposite to the above side.

The guide 222 may have a shape that is gradually narrowed from one side connected to the outer circumference part 223 toward the other side connected to the coupling part 221. In other words, the guide 222 may be provided as an inclined surface connecting the outer circumference part 223 to the coupling part 221.

When the driving module 100 is inserted in the first housing 210 through the guide assembly 220, one side of the guide 222, which is connected to the outer circumference part 223, may be an inlet where the insertion of the driving module 100 starts and the other side of the guide 222, which is connected to the coupling part 221, may be an outlet through which the driving module 100 exits.

The guide assembly 220 according to an embodiment of the present disclosure has the inlet, where the insertion of the driving module 100 starts, to be relatively wide, and the outlet, through which the driving module 100 discharges, to be relatively narrow. Thus, during the insertion, the centers of the driving module 100 and the first housing 210 may be aligned, and the insertion path may be guided to mount the driving module 100 at the precise location in the first housing 210.

Also, the guide assembly 220 according to an embodiment of the present disclosure guides the insertion path of the driving module 100 to avoid contact with the regions other than the inside of the handle case 200 when inserting the driving module 100 of the non-sterilized state, and accordingly, contamination of the handle case 200 that is in the sterilized state may be prevented.

Also, the guide assembly 220 according to an embodiment of the present disclosure may be easily separated from the first housing 210 after finishing the mounting of the driving module 100, and thus, the user's convenience in use may be improved.

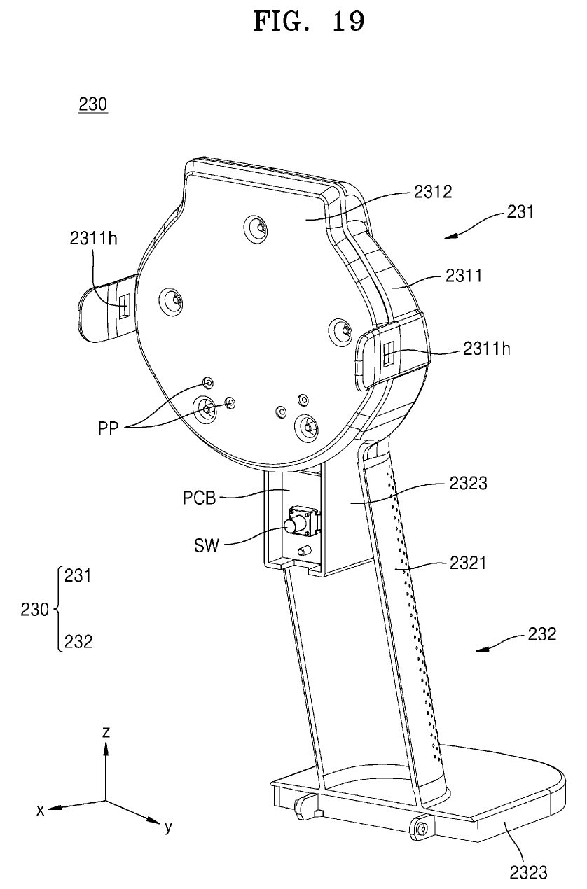

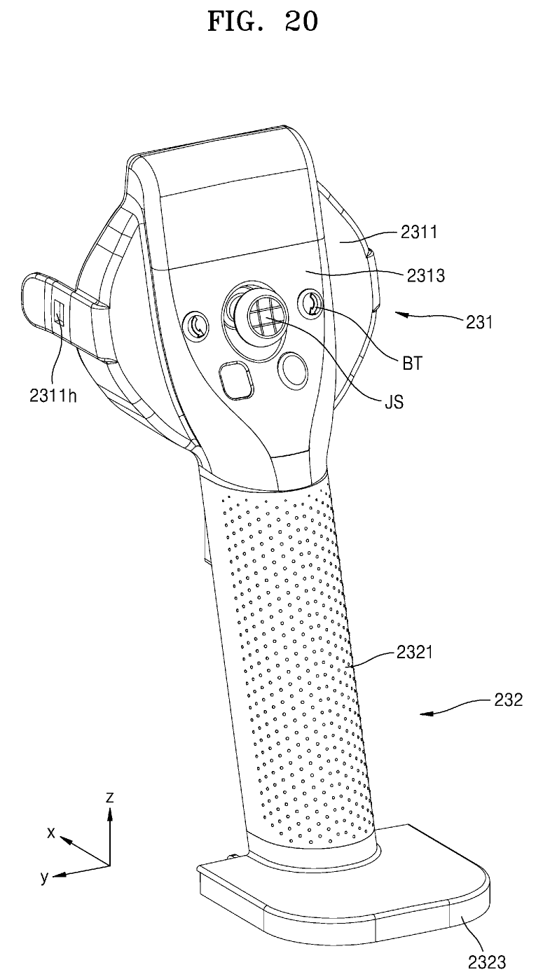

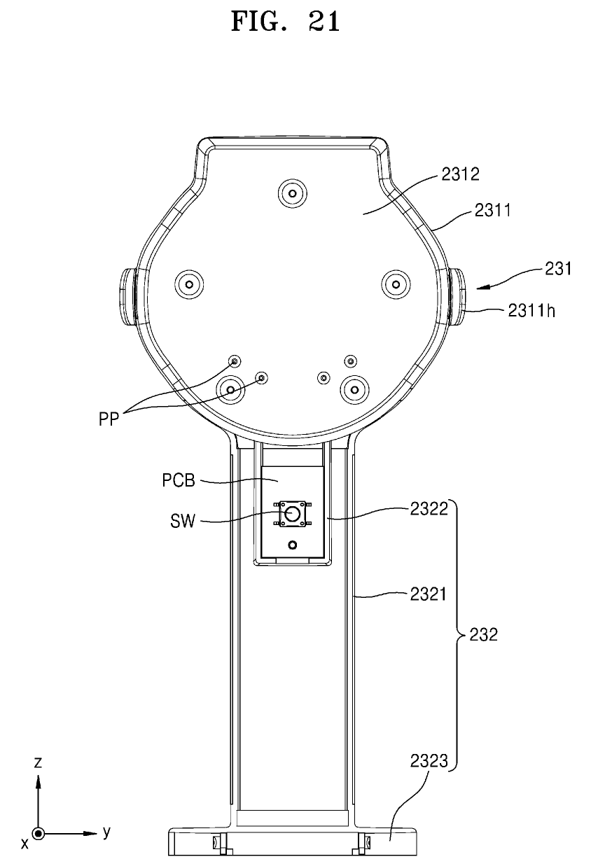

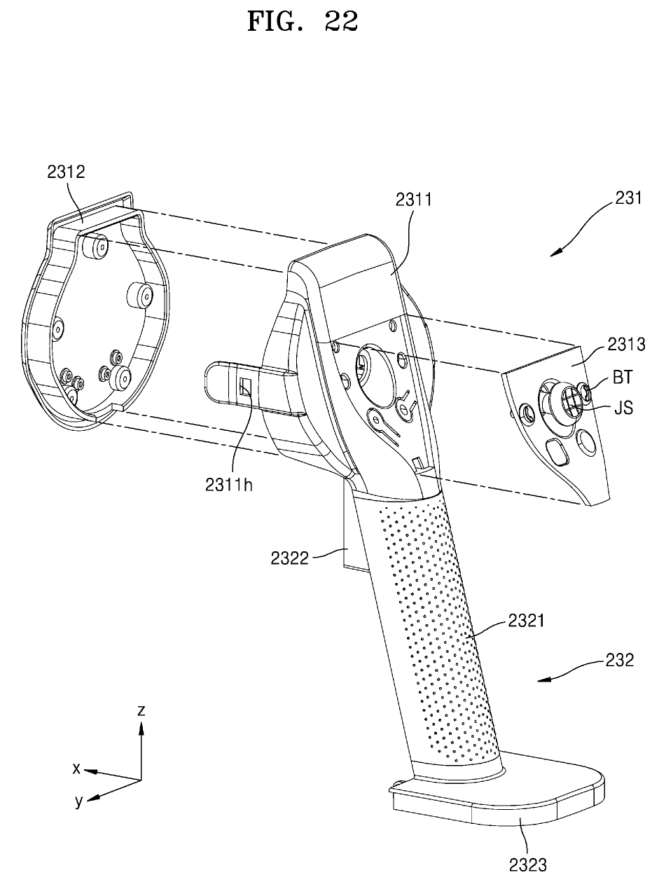

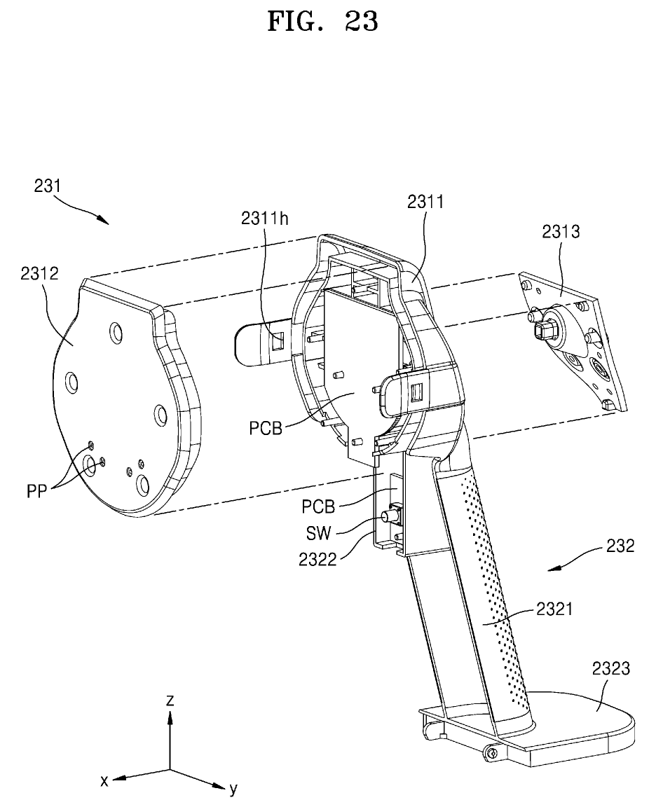

FIG. 19 is a perspective view of a second housing according to an embodiment of the present disclosure. FIG. 20 is a perspective view of the second housing from another direction, according to an embodiment of the present disclosure. FIG. 21 is a front view of the second housing according to an embodiment of the present disclosure. FIG. 22 is an exploded perspective view of a second housing according to an embodiment of the present disclosure. FIG. 23 is an exploded perspective view of the second housing from another direction, according to an embodiment of the present disclosure.

Referring to FIGS. 19-23, the second housing 230 may include a cover 231 and a second handle assembly 232. The second housing 230 may be coupled to the first housing 210 while the guide assembly 220 is separated from the first housing 210.

The second housing 230 may be provided with an input device (joystick, button, etc.) capable of transmitting a signal for driving the end tool 10.

Pogo pin PP structures may be disposed in one surface of the second housing 230, which faces the driving module 100 while the handle case 200 is closed. The pogo pins PP may transmit the electrical signal input from the input device to the driving module 100 by coming into contact with contact points disposed on the rear surface of the driving module 100.

That is, the second housing 230 may be provided with the input device on one side thereof and the pogo pins PP on the other side facing the above side, and the signal input from the input device may be transmitted to the driving module 100 via a printed circuit board (PCB) in the second housing 230 and the pogo pins PP.

The cover 231 may cover one open end of the first housing 210 and may include a cover body 2311, an inner case 2312, and an outer case 2313.

The cover body 2311 receives the PCB therein and is connected to the first housing 210, a stopper hole part 2311h that is recessed toward the first housing 210 and may be coupled to the stopper protrusion 211p may be formed in one side of the cover body 2311.

Either the guide assembly 220 or the second housing 230 may be coupled to the stopper protrusion 211p of the first housing 210. That is, when mounting the driving module 100, the guide assembly 220 may be coupled to the stopper protrusion 211p, and when the mounting of the driving module 100 is finished, the second housing 230 may be coupled to the stopper protrusion 211p.

A plurality of stopper hole parts 2311h may be provided and may be arranged in both sides of the cover body 2311 to be respectively coupled to the plurality of stopper protrusions 211p disposed on the first housing 210.

The printed circuit board PCB that physically fixes and electrically connects the electronic components may be arranged in the cover body 2311. An MCU capable of converting an analog signal input from the outside into a digital signal may be mounted on the printed circuit board PCB. Moreover, various resistors, capacitors, the switch SW, etc. may be mounted on the printed circuit board PCB.

The inner case 2312 seals the open end of the first housing 210 and may come into contact with the driving module 100. In detail, the inner case 2312 is fixed to the cover body 2311, and when the second housing 230 is rotated and seals the open end of the first housing 210, the inner case 2312 may come into contact with at least a part of the inner circumference of the first housing 210.

At least a part of the inner case 2312 may be formed of a material having elastic recovery force.

The inner case 2312 may have a shape corresponding to the open end of the first housing 210. At least a part of the inner case 2312 may be formed as a circular shape and another part may protrude toward the outside (upper side in FIG. 23). Therefore, while the handle case 200 is closed, the inner case 2312 may all cover the open end of the first housing 210 and the raised part 211a.

The outer circumference defined along the edge of the inner case 2312 may be an inclined surface so as to be able to contact the inner circumference of the first housing 210. As such, a part of the inner case 2312 may be easily inserted into the open end of the first housing 210.

The inner case 2312 having the elastic recovery force comes into contact with the inner circumference of the first housing 210 when being inserted into the first housing 210, and may be pushed by the inner circumference of the first housing 210 and partially deformed. In the above state, the first housing 210 may be sealed. While the handle case 200 is closed, the inner case 2312 may be arranged between the driving module 100 and the cover body 2311.

The inner case 2312 seals the open end of the first housing 210, and at the same time, also seals the open end of the cover body 2311. Thus, the driving module 100 and the printed circuit board PCB may be prevented from coming into contact with the outer contamination substances, dust, moisture, etc.

A plurality of pogo pins PP that may come into contact with the contacts set in advance on the driving module 100 may be arranged on the inner case 2312. When the handle case 200 in which the driving module 100 is mounted is closed, the pogo pins PP may come into contact with the rear surface of the driving module 100 and transmit electrical signals to the driving module 100.

The outer case 2313 is arranged to face the inner case 2312 and may be provided with an interface for manipulating the driving of the end tool 10. For example, the outer case 2313 may have various buttons BT, joystick JS, etc. through which the signal for controlling motions of the end tool 10 is input.

One side of the joystick JS may pass through the outer case 2313 and may come into contact with the printed circuit board PCB arranged inside the cover body 2311. The button BT may be arranged so as to come into contact with the switch SW installed on the printed circuit board PCB. When the user exerts the force to the button BT for inputting signals, the switch SW is pushed and may transmit the input signal for driving the end tool 10.

The interface is driven when being applied with the power from the driving module 100 and may convert a manipulation signal input from the outside into the electrical signal and transmit the electrical signal to the driving module 100.

Because the handle case 200 according to an embodiment of the present disclosure has no separate supply source for supplying electric power, the power from the driving module 100 has to be used in order to process the input signal.

Therefore, the interface receiving the input of the analog signal from the outside and the MCU capable of converting the analog signal into the digital signal may be driven by the electric power from the driving module 100. The converted signal is transmitted to the driving module 100 again, and as such, various motions such as a tilting motion, a pivoting motion, etc. of the end tool 10 may be implemented.

The second handle assembly 232 according to an embodiment of the present disclosure may extend in the longitudinal direction and may be gripped by the user, and may include a second handle 2321, an accommodation part 2322, and a second support 2323.

The second handle 2321 extends in the longitudinal direction and may be a region held by the hand of the user when using the handle case 200. The second handle 2321 may extend downward (based on FIG. 19) and may be arranged to have an angle of at least 90° with respect to the center axis direction of the housing body 211 while the handle case 200 is closed.

The second handle 2321 may be formed as a half of a hollow cylinder cut in halves. The second handle 2321 may form a cylindrical shape by coupling to the first handle 2121, and the cylindrical shape may be arranged to be inclined to form an angle set in advance with respect to the second support 2323.

The second handle 2321 may be a region directly gripped by the user when driving the end tool 10. A plurality of protrusions (not indicated by reference numerals) protruding outward may be disposed on the surface of the second handle 2321. The plurality of protrusions may be arranged to be spaced apart from one another with preset intervals, and provide the user with stabilized grip to prevent slipping during use when gripped by the user.

The accommodation part 2322 may be at least partially surrounded by the second handle 2321 and protrude from the second handle 2321 to have an internal space. The accommodation part 2322 may be arranged closer to the cover 231 than the second support 2323.

The switch SW generating the electrical signal for driving the end tool 10 may be arranged in the internal space of the accommodation part 2322.

The second support 2323 is arranged to form an angle set in advance with respect to the second handle 2321 and has a plate shape, and may extend in a direction away from the first housing 210.

The second support 2323 may be connected to the first support 2123 in a hinged manner. Therefore, when the guide assembly 220 is mounted in the first housing 210, the second support 2323 may be arranged to form an angle set in advance with respect to the first support 2123. When the guide assembly 220 is removed, the second support 2323 rotates and then may be arranged on the same plane as the first support 2123.

In other words, the first support 2123 and the second support 2323 may form one plane and support the first handle 2121 and the second handle 2321.

Referring back to FIG. 6, when the second housing 230 is coupled to the first housing 210, a center of weight of the surgical instrument 1 may be arranged to be closer to the side E1 of the handle case 200, to which the adaptor is coupled, than the side E2 of the handle case 200 gripped by the user. In this case, the handle case 200 may lean toward the side E1.

The second support 2323 according to an embodiment of the present disclosure may be arranged at the outermost side of the surgical instrument 1 as being extended in the direction away from the first housing 210, and accordingly, when the user grips the handle case 200, the second support 2323 supports the edge of the hand of the user and prevents the handle case 200 from leaning to one side, thereby providing a stabilized feeling in use.

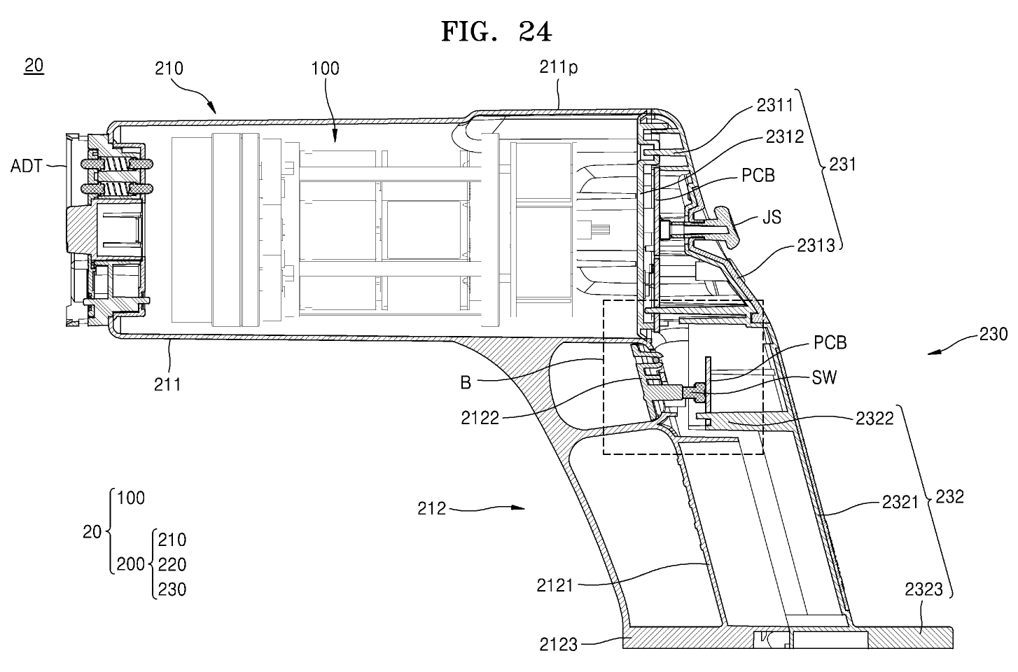

FIG. 24 is a side-sectional view of a manipulation part while a handle case of the present disclosure is closed. FIG. 25 is an enlarged view of part B of FIG. 24.

Referring to FIGS. 24 and 25, the first support 2123 and the second support 2323 may be arranged on the same plate as each other. The first handle 2121 and the second handle 2321 are coupled to each other and may have a cylindrical shape that is inclined by a certain angle with respect to the plane.

At least a part of the accommodation part 2322 may be arranged inside the first handle 2121.

The accommodation part 2322 may be arranged to face the trigger 2122 passing through at least a part of the first handle 2121. The switch SW arranged in the internal space of the accommodation part 2322 may be arranged to be able to come into contact with the input transmission part 2122a that passes through the through-hole 2121h and protrudes in the direction toward the second handle 2321.

When the user has the finger on the trigger 2122 and pulls the trigger 2122, the input transmission part 2122a may exert the force to the switch SW in the direction toward the second handle 2321, and the switch SW may generate the electrical signal for driving the end tool 10. The electrical signal generated by the switch SW may be transmitted to the driving module 100 via the printed circuit board PCB.

When the user physically tilts or rotates the joystick JS arranged on the outer case 2313 to the up and down/left and right sides, the physical movement manipulated by the user may be converted into the electrical signal. When the electrical signal that is converted is transmitted to the driving module 100 through the pogo pins PP of the inner case 2312 via the printed circuit board PCB, the tilting or rotating motion of the end tool 10 may be implemented.

According to the embodiment of the present disclosure, the handle case of the surgical instrument used in the laparoscopic surgery or other various surgeries is provided with the guide assembly, and accordingly, the driving module may be easily mounted and the convenience in use of the handle case may be improved.

While the present disclosure has been particularly shown and described with reference to preferred embodiments thereof, it will be understood by those skilled in the art that various changes in form and details may be made therein without departing from the spirit and scope of the present disclosure as defined by the appended claims. The preferred embodiments should be considered in descriptive sense only and not for purposes of limitation. Therefore, the concept of the present disclosure is defined not by the detailed description of the present disclosure but by the appended claims, and all differences within the scope will be construed as being included in the present disclosure.

What is claimed is:

1. A handle case mountable on a surgical instrument,
the handle case comprising:
a first housing having an accommodation space configured to accommodate a driving module, and an open end through which the driving module enters the accommodation space;
a second housing configured to cover the open end of the first housing; and
a guide assembly detachably disposed at the open end of the first housing and configured to guide an insertion path of the driving module.

2. The handle case of claim 1, wherein the first housing comprises:
a housing body in which the driving module is configured to be mounted; and
a first handle assembly disposed at a predetermined angle with respect to the housing body and configured to be gripped by a user.

3. The handle case of claim 2, wherein the first handle assembly and the housing body are disposed to form an obtuse angle.

4. The handle case of claim 1, wherein the guide assembly comprises:
a coupling part configured to be coupled to the open end of the first housing; and
a guide connected to the coupling part and inclined in a direction toward an inside of the first housing.

5. The handle case of claim 4, wherein the coupling part has a dual inner wall structure including a first inner wall and a second inner wall disposed along a circumference of the coupling part and spaced apart by a predetermined interval, and
a portion of the first housing is inserted between the first inner wall and the second inner wall.

6. The handle case of claim 1, wherein the second housing comprises:
a cover configured to cover the open end of the first housing; and
a second handle assembly extending from the cover and configured to be gripped by a user.

7. The handle case of claim 6, wherein the cover comprises:
a cover body accommodating a printed circuit board therein and connected to the first housing; and
an inner case configured to seal the open end of the first housing and contact the driving module.

8. The handle case of claim 7, wherein
an outer circumferential surface defined along an edge of the inner case is an inclined surface disposed to contact an inner circumference of the first housing.

9. The handle case of claim 7, wherein
the cover further comprises an outer case disposed to face the inner case and having an interface configured to manipulate a driving of the driving module.

10. The handle case of claim 9, wherein
the interface is configured to operate by receiving electric power from the driving module, convert a manipulation signal that is input from outside into an electrical signal, and transmit the electrical signal to the driving module.

11. The handle case of claim 6, wherein the second handle assembly comprises:
a second handle extending in a longitudinal direction; and
a second support disposed at a predetermined angle relative to the second handle and having a plate shape.

12. A surgical instrument comprising:
an end tool including one or more jaws configured to rotate; and
a manipulation part including a driving module configured to generate power to drive the end tool, and a handle case accommodating the driving module and configured to be gripped by a user,
wherein the handle case comprises a first housing defining an accommodation space configured to accommodate the driving module and having an open end through which the driving module enters the accommodation space, and a second housing covering the open end of the first housing.

13. The surgical instrument of claim 12, wherein
the accommodation space has a shape corresponding to a cylindrical shape of the driving module extending in a longitudinal direction, and
the accommodation space is enclosed from outside when the second housing is coupled to the first housing.

14. The surgical instrument of claim 12, wherein the first housing comprises:
a housing body in which the driving module is mounted; and
a first handle assembly disposed at a predetermined angle with respect to the housing body and configured to gripped by the user.

15. The surgical instrument of claim 14, wherein the first handle assembly and the housing body are disposed to form an obtuse angle.

16. The surgical instrument of claim 12, wherein the second housing comprises:
a cover configured to cover the open end of the first housing; and
a second handle assembly extending from the cover and configured to be gripped by the user.

17. The surgical instrument of claim 16, wherein the cover comprises:
a cover body accommodating a printed circuit board therein and connected to the first housing; and
an inner case configured to seal the open end of the first housing and contact the driving module.

18. The surgical instrument of claim 17, wherein
an outer circumferential surface defined along an edge of the inner case is an inclined surface disposed to contact an inner circumference of the first housing.

19. The surgical instrument of claim 17, wherein
the cover further comprises an outer case disposed to face the inner case and having an interface configured to manipulate a driving of the driving module.

20. The surgical instrument of claim 19, wherein
the interface is configured to operate by receiving an application of electric power from the driving module, convert a manipulation signal that is input from outside into an electrical signal, and transmit the electrical signal to the driving module.
